(19) United States
(12) Patent Application Publication
(10) Pub. No.: US 2007/0212766 A1
(43) Pub. Date: Sep. 13, 2007

(54) METHOD FOR THE ENZYMATIC PRODUCTION OF CHIRAL ALCOHOLS

(75) Inventors: Rupert Pfaller, Muenchen (DE); Jurgen Stohrer, Pullach (DE)

Correspondence Address: BROOKS KUSHMAN P.C., 1000 TOWN CENTER, TWENTY-SECOND FLOOR, SOUTHFIELD, MI, 48075, US

(73) Assignee: WACKER CHEMIE AG

(21) Appl. No.: 11/683,120

(22) Filed: Mar. 7, 2007

(30) Foreign Application Priority Data
Mar. 9, 2006 (DE) 102006010994.5

(51) Int. Cl. C12P 7/04

(52) U.S. Cl. 435/157

(57) ABSTRACT

A method of producing a chiral secondary alcohol in which a biotransformation composition containing a ketone of the formula (I),

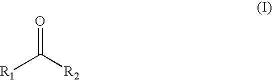

R₁ and R₂ being different and each being an organic radical, an oxidoreductase and a co-substrate is reacted to form a chiral secondary alcohol with the adsorbent being associated with the oxidoreductase. The adsorbent associated with the oxidorecutase is separated off from the biotransformation composition after completion of the reaction.

### BACKGROUND OF THE INVENTION

1. Field of the Invention

The present invention relates to a method for the efficient enzymatic production of chiral alcohols.

2. Background Art

Optically active hydroxyl compounds are valuable synthesis building blocks, for example in the production of active pharmaceutical ingredients or of agrochemicals. These compounds can frequently only be produced with difficulty by classical chemical methods, since the required optical purities for uses in the pharmaceutical and agrochemical sector can be achieved only with difficulty in this manner. Therefore, for the production of chiral compounds, biotechnological methods are being used to an increasing extent. Especially enzymes which can reduce carbonyl compounds are increasingly being used because of their high enantioselectivity.

Enzymes of the class of oxidoreductases which are used for producing chiral compounds by reduction of prochiral carbonyl compounds are designated by the collective term carbonyl reductase (hereinafter “CR”). In the majority of cases the product of a CR reaction is an alcohol. However, it is also possible that the product of a CR reaction is an amine. The carbonyl reductases include, inter alia, alcohol dehydrogenases (hereinafter “ADH”), aldo-keto reductases (“AKR”), aldehyde reductases, glycerol dehydrogenases and fatty acid synthase (designated “FAS”). However, amino-transferases or amino acid dehydrogenases (for example threonine dehydrogenase) also belong to the carbonyl reductases. This broad spectrum of reducing enzymes share the fact that they obtain the electrons for reducing the carbonyl compound from redox cofactors in their reduced form, customarily NADH or NADPH.

The redox cofactors NADH and NADPH are consumed stoichiometrically in the enzymatic reduction, that is they must either be used stoichiometrically or else regenerated by oxidation of a co-substrate (termed cofactor regeneration). A co-substrate in this case is defined as a compound which is oxidized enzymatically as reducing agent, with the electrons produced in this process being transferred to NAD or NADP and NADH or NADPH thereby being regenerated.

These biotransformation processes in most cases have the disadvantage of a low economic efficiency, in that the enzyme usage is very high and the space-time yields are low. Moreover, the economic efficiency of a biotransformation is frequently impaired by the fact that either the starting material or the product leads to inactivation of the enzyme.

A review of industrially utilized biotransformations is given by Breuer et al. (2004), Angew. Chem. 116: 806-843.

Both enzyme usage and space-time yield are critical cost factors which determine the economic efficiency of a biotransformation. The prior art currently does not provide a generally usable method by which chiral alcohols can be produced inexpensively.

To ensure efficient enzyme usage, it is in principle possible to recover the enzyme from a biotransformation batch by extracting the reaction batch, the enzyme remaining in the aqueous reaction phase. A precondition for this, however, is high stability of the enzyme versus organic solvents. EP 1568780 discloses a method for recovery and reuse of the enzyme LB-ADH in the reduction of methyl acetoacetate to methyl (R)-3-hydroxybutyrate, although in the case of low usage of starting material of 5% (w/v).

Increasing the space-time yield is another possibility for improving the economic efficiency of biotransformations. DE102005038606 (example 3) discloses the production of (R)-1-acetoxy-2-propanol from acetoxyacetone at a high starting material addition of 43% (w/v), but with high enzyme usage without the possibility of enzyme recovery. To improve the economic efficiency of biotransformations, a method would be desirable which combines high space-time yields with an efficient enzyme usage.

Adsorbents are widely used in biotechnological methods. They are used, inter alia, as filtration aids in order to remove suspended solids. They are in addition used in chromatography for separation of substances and are also used as support materials.

In enzymatic synthesis, the use of support materials is known especially for immobilizing enzymes. The immobilization of whole cells in alginate has also been described, and the effect of enantioselectivity in the presence of an adsorbent. The best-known example of a support material for immobilization of enzymes is Eupergit® from Röhm. Those that are commercially available are primarily covalently immobilized lipases (for example obtainable from Novozymes), which can be used for racemate resolution. The production of immobilized enzymes is, however, a complex process so that their use is customarily not connected with significant cost savings. Covalently immobilized enzymes are also unsuitable for improving the economic efficiency of the synthesis of chiral alcohols.

Thus, to date, no simple and inexpensive method is known which makes it possible for those skilled in the art, by using adsorbents, to improve the economic efficiency of the production of chiral alcohols by enzymatic reduction with respect to enzyme usage and space-time yield.

### SUMMARY OF THE INVENTION

It is an object of the invention to provide a method, which enables efficient inexpensive production of a chiral secondary alcohol. The present object is achieved by a method in which a biotransformation composition containing a ketone of the formula (I),

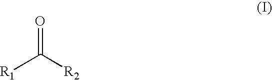

R₁ and R₂ being different and each being an organic radical, an oxidoreductase, and a co-substrate are reacted to form a chiral secondary alcohol. The biotransformation composition contains an adsorbent, which associates with the oxidoreductase, and which is separated off from the biotransformation composition after the reaction is completed.

### BRIEF DESCRIPTION OF THE DRAWINGS

FIG. 1 is a vector map of the expression plasmid petAKRgd.

### DETAILED DESCRIPTION OF THE PREFERRED EMBODIMENT(S)

In an embodiment of the present invention, a method enabling efficient inexpensive production of a chiral secondary alcohol is provided. The method of this embodiment comprises reacting a biotransformation composition containing a ketone of the formula (I),

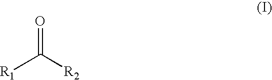

R₁ and R₂ being different and each being an organic radical with an oxidoreductase and a co-substrate to form a chiral secondary alcohol. The biotransformation composition contains an adsorbent, which associates with the oxidoreductase. After the reaction is completed, the adsorbent is separated off from the biotransformation composition.

In accordance with the method of this embodiment, the oxidoreductase is recovered from the biotransformation composition and is reusable for the biotransformation of a ketone of the formula (I) to a chiral secondary alcohol. The present method makes it possible to minimize the enzyme usage in biotransformations by recycling, at high space-time yields also.

As adsorbent, all solids are suitable which are able to keep the oxidoreductase in active form during the biotransformation reaction and, after termination of the biotransformation, when the adsorbent is separated off from the reaction batch, preferably by filtration, sedimentation or centrifugation, to retain the oxido-reductase in the filter cake in active form. As a result, the enzyme is separated off from the reaction batch and it can be recycled for reuse in a new reaction batch (enzyme recycling).

Owing to the simple process procedure, adsorbents are preferred which bind the oxidoreductase noncovalently. The noncovalent binding can proceed in this case by hydrophobic or electrostatic interaction (ion exchanger).

Suitable adsorbents include inorganic and organic materials. Suitable adsorbents based on inorganic compounds include aluminum oxides and aluminum oxide hydrates, such as, for example, calcined aluminum hydroxide, silicon dioxide and silicic acids, for example diatomaceous earths such as Celite®, precipitated silicic acids such as, for example, silica gel, aerogel or fumed silicas such as HDK®. Suitable inorganic adsorbents also include silicates, including aluminosilicates, such as, for example, zeolites, magnesium silicates such as, for example, Florisil® or talcum, calcium silicates such as, for example, calcium metasilicate and also mixed silicates such as, for example, bentonites. Suitable inorganic adsorbents include, in addition, calcium phosphates such as, for example, hydroxyapatite.

Suitable adsorbents based on organic compounds include cationic and anionic ion exchangers based on polystyrene resins such as, for example, XAD, Dowex® and Amberlite®. Additional organic adsorbents include polysaccharides, for example cellulose and crosslinked dextrans such as, for example, Sephadex®, Sephacryl® or Sephacel®, furthermore crosslinked agarose such as, for example, Sepharose® and Superose®. The organic adsorbents in addition include those based on activated carbons and methacrylates. Depending on the surface nature, these adsorbents can be suitable for hydrophobic or electrostatic interaction.

Suitable adsorbents are found in the product catalogues of relevant manufacturers such as, Merck, Fluka, Röhm, Rohm und Haas, Sigma-Aldrich, Supelco (product catalogue “Chromatographie: Produkte für die Analytik und Aufreinigung” [Chromatography: Products for analysis and purification], 2006-2007, pp. 561-602) or GE Healthcare (product catalogue “Products for Life Sciences”, 2006, pp. 506-636). Preferred adsorbents are XAD, Florisil®, Dowex®, Amberlite®, silica gel, Celite®. A particularly preferred adsorbent is Celite®.

Although said adsorbents are already used as described in the prior art in biotechnological methods, these uses give no indication, however, of the method according to the invention. In particular, it is completely unexpected that by using the adsorbents it is possible to recover the functional enzyme from the biotransformation composition, although these compositions have a fraction of organic compounds of 50-80% (v/v) (composed of starting material, co-substrate and any solvent), which is unusually high for enzymatic reactions.

Preferably, the chiral secondary alcohol is a compound of the formula (II) or (III)

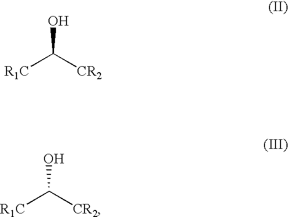

wherein R₁ and R₂ are an organic radical and are different from one another. Preferably, R₁ and R₂ are different organic radicals having 1-20 carbon atoms. Examples of preferred radicals include unbranched or branched C₁-C₂₀-alkyl, C₂-C₂₀-alkenyl, C₂-C₂₀-alkynyl, C₃-C₈-cycloalkyl, C₆-C₂₀-aryl or C₅-C₂₀-heteroaryl radicals. One or more carbon atoms of the radicals R₁ or R₂ may be replaced by atoms selected from the group B, N, O, Si, P and S, or by F, Cl, Br, I, or by optionally substituted C₃-C₈-cycloalkyl, C₆-C₂₀-aryl, C₅-C₂₀-heteroaryl, or by silyl radicals and also by CN, NH₂, NO or NO₂. More preferably, R₁ and R₂ are selected from the group C₁-C₁₂-alkyl, C₁-C₁₂-alkenyl, C₂-C₁₂-alkynyl, C₃-C₈-cycloalkyl, C₆-C₂₀-aryl or C₅-C₂₀-heteroaryl radical in which one or more carbon atoms can be substituted by F, Cl, C₃-C₈-cycloalkyl, C₆-C₂₀-aryl or C₅-C₂₀-heteroaryl, or by atoms selected from the group N, O and S.

The oxidoreductase is preferably a CR having S or R specificity. Secondary ADHs are preferred when R-specific CRs are used. Example of such secondary ADH's are from strains of the genus Lactobacillus, such as the ADHs from Lactobacillus brevis (LB-ADH), Lactobacillus kefir, Lactobacillus parabuchneri, Lactobacillus kandleri, Lactobacillus minor, or of fatty acid synthetases (FAS). The FAS of baker's yeast or from Pichia pastoris is more preferred. Preferred R-selective CRs are ADHs from the genus Lactobacillus. A most preferred R-selective CR is LB-ADH.

As S-specific CRs, use is preferably made of Secondary ADHs. Examples of such secondary ADHs are from strains of the genus Thermoanaerobacter (Thermoanaerobium) such as the ADHs from Thermoanaerobacter sp. (T-ADH, disclosed in DE 102004029112 A1), Th. brockii or Th. ethanolicus, or from strains of the genus Rhodococcus such as the ADHs from Rhodococcus ruber (RR-ADH) or Rhodococcus erythropolis (RE-ADH). CRs from baker's yeast (examples of S-specific CRs from baker's yeast are disclosed in Kaluzna et al. (2004), J. Am. Chem. Soc. 126: 12827-12832) may also be used. Preferred S-selective CRs are ADHs from the genera Thermoanaerobacter and Rhodococcus. More preferably, S-selective CRs are T-ADH and RR-ADH. An in particular preferred S-selective CR is T-ADH.

The CRs used for enzymatic reduction can be produced by culturing the microorganism from which the CR in question originates. This is achieved in each case in a manner known to those skilled in the art. The CR enzyme produced in this manner can be used directly in the cells of the production host. Alternatively, it can be used after digestion of the cells as protein extract, or as purified protein after corresponding workup by, for example, column chromatography. The enzyme production of the CRs can proceed using an expression system, also in recombinant form. For this, the gene coding for the CR in question is isolated and, in accordance with the prior art, cloned into an expression vector suitable for protein production. After transformation of the expression vector into a suitable host organism, a production strain is isolated. The CR may be produced in a manner known per se using this production strain, for example by fermentation. The CR enzyme produced in this manner can then be further used directly in the cells of the production host, or after digestion of the cells, as protein extract, or as purified protein after appropriate workup (for example, column chromatography). Preferably, the enzyme production of the CRs of the invention use an expression system in recombinant form. For enzyme production, bacterial and eukaryotic expression systems are suitable. Host organisms for enzyme production are preferably selected from Escherichia coli, strains of the genus Bacillus, yeasts such as Pichia pastoris, Hansenula polymorpha or Saccharomyces cerevisiae and also fungi, such as Aspergillus or Neurospora. However, such systems are not restricted to said host organisms. The preferred expression systems include E. coli, Bacillus, Pichia pastoris, S. cerevisiae, Hansenula polymorpha or Aspergillus. More preferably, systems for production of the CR enzyme are E. coli, Pichia pastoris and S. cerevisiae. A particularly preferred expression system is E. coli.

To achieve enzyme cost-efficient usage, the enzyme production preferably proceeds by fermentation, more preferably in a fed-batch method. Preferably, the cells from the fermentation (fermenter cells) are then directly used, suspended in the fermentation medium or after isolation in advance and subsequent resuspension, in the method of the invention, so that the method of the invention is conducted as whole-cell biotransformation. It is also possible, however, in the method of the invention, after disruption of the cells, to use the resultant protein extract, or after appropriate workup (for example, column chromatography) to use the resultant purified protein in the method of the invention.

Whole-cell biotransformation in which first the enzyme production proceeds in a recombinant host cell by means of fermentation are preferred. In such biotransformations, the fermenter cells are subsequently directly used, suspended in the fermentation medium or after isolation in advance and subsequent resuspension.

The biotransformation composition may comprise a redox cofactor. The redox cofactor is a compound, which in its reduced form provides electrons which are transferred in the enzymatic reaction by an oxidoreductase to the starting material with the result that a product of the invention is formed. The redox cofactor is preferably selected from compounds of the group NAD, NADP, (in each case oxidized form of the cofactor), NADH, NADPH (in each case reduced form of the cofactor) and salts thereof. The redox cofactors in their reduced form, NADH and NADPH, are consumed stoichiometrically in the enzymatic reduction, that is they must either be used stoichiometrically or else regenerated by oxidation of a co-substrate (cofactor regeneration). Stoichiometric usage of NADH or NADPH is uneconomical because of the high price of these compounds. This disadvantage is circumvented by cofactor regeneration. A precondition for this is an inexpensive co-substrate (reducing agent) and a cofactor-reducing enzyme. Efficient and inexpensive regeneration of the redox cofactor first makes possible the industrial use of biocatalytic reduction methods.

The use of an adsorbent permits the simple separation of the enzyme from the reaction batch by, for example, filtration, sedimentation or centrifugation, and its reuse in a new biotransformation composition. Any enzyme lost in the recovery is able to be restored in the new batch in order to maintain the maximum conversion rate. In this manner, in principle, it is possible to carry out an unlimited number of reaction cycles.

A co-substrate is a compound which is enzymatically oxidized as reducing agent. The electrons produced in this case are transferred to NAD or NADP thereby regenerating NADH or NADPH.

If in the method of the invention a CR of the class of ADHs is used, an alcohol is used as co-substrate for the cofactor regeneration. Preferably, such alcohols are inexpensive (e.g., isopropanol or 2-butanol). However, all other higher secondary alcohols derived from 2-butanol are also suitable. Accordingly, this method variant provides not only stereoselective reduction of the starting material but also cofactor regeneration by the same enzyme, the ADH.

If in the method according to the invention a CR which is not an ADH is used, the cofactor is regenerated by means of a second enzyme. This is likewise situated in the reaction batch. The CR reduces the starting material stereoselectively to give the desired product, with the cofactor NADH or NADPH being consumed. The regeneration of the consumed NADH or NADPH is performed by a second enzyme. In principle, any enzyme, which oxidizes a substrate in one enzymatic reaction and simultaneously reduces NAD to NADH or NADP to NADPH is suitable for cofactor regeneration. Preferably, use is made of an enzyme which oxidizes a inexpensive co-substrate, such as, for example, glucose or formic acid, or salts thereof. Preferably, as enzyme for cofactor regeneration, use is made of an enzyme from the group glucose dehydrogenase (GDH) and formate dehydrogenase (FDH).

Preferred combinations of enzyme/co-substrate for cofactor regeneration are the combination of an ADH with an alcohol such as, for example, isopropanol or 2-butanol, or the combination of a GDH with glucose. More preferably, the combination of an ADH with an alcohol such as, for example, isopropanol or 2-butanol is used. Most preferably, the combination of an ADH with isopropanol is used.

The method according to the invention makes it possible, by enzymatic reduction of a starting material of the formula (I), to produce chiral secondary alcohols by means of a simple batch method at high space-time yields and with low enzyme usage.

In the simplest form a biotransformation composition of the invention (a batch) comprises fermenter cells containing a CR enzyme, an adsorbent, as starting material a compound of the formula (I), a redox cofactor selected from the compounds NAD, NADH, NADP, NADPH, and salts thereof, a co-substrate selected from the group isopropanol, 2-butanol and glucose, and also, when glucose is used as co-substrate, a GDH as cofactor-regenerating enzyme.

In a modified form of the method, one or more of said components of the biotransformation composition are added continuously or batchwise (fed-batch method). Preferably, the method is the batch method.

The CR enzyme used in the method according to the invention can be contained in whole cells (whole-cell method). Alternatively, purified protein obtained after digestion of the cells, as protein extract, or after appropriate workup (for example, column chromatography) is used in the biotransformation composition of the invention. Preferably, the CR enzyme in a whole-cell method is used.

Preferably, a biotransformation composition of the invention contains between 1% (v/v) and 40% (v/v) of a suspension of fermenter cells obtained from the fermentation having a biomass fraction of 0.05-2% (w/v), containing a CR enzyme. The biomass fraction is defined in this case as dry biomass which is obtained when the fermenter cells are dried to constant weight for example in a drying cabinet at 105° C. More preferably, the composition contains between 5% (v/v) and 30% (v/v) of a suspension of fermenter cells obtained from the fermentation containing a CR enzyme having a biomass fraction of 0.25-1.5% (w/v). Most preferably, the composition contains between 10% (v/v) and 25% (v/v) of a suspension of fermenter cells obtained from the fermentation containing a CR enzyme having a biomass fraction of 0.5-1.25% (w/v). In addition, a biotransformation composition of the invention contains between 0.1% (w/v) and 10% (w/v) of an adsorbent. Preferably, the composition contains between 0.2% (w/v) and 5% (w/v) of an adsorbent. Particularly preferably, the composition contains between 0.5% (w/v) and 3% (w/v) of an adsorbent.

Use of the adsorbent permits simple separation of the enzyme from the reaction batch by, for example, filtration, sedimentation or centrifugation and its reuse in a new biotransformation composition, with any enzyme lost in the recovery being able to be restored in the new batch, in order to maintain the maximum conversion rate. In this manner it is possible in principle to carry out an unlimited number of reaction cycles. Preferably, up to 20 reaction cycles are carried out in the method of the present invention. More preferably, up to 4 reaction cycles are carried out in the method of the present invention.

The CR enzyme can be present in whole cells. Alternatively, protein extract or purified protein after appropriate workup (for example, column chromatography) obtained after digestion of the cells can be used in the biotransformation composition of the invention.

A biotransformation composition of the invention is further distinguished by the fact that the fraction of starting material of the formula (I) is preferably between 5% (w/v) and 60% (w/v) of the total batch. More preferably, the fraction of starting material of the invention of the formula (I) is between 20% (w/v) and 50% (w/v) of the total batch. Most preferably, a composition in which the fraction of starting material of the formula (I) is between 30% (w/v) and 45% (w/v) of the total batch is used.

A biotransformation composition of the invention is also distinguished by the fact that the fraction of co-substrate in the case of isopropanol or 2-butanol is preferably between 10% (w/v) and 50% (w/v) of the total batch. More preferably, the fraction of co-substrate in the case of isopropanol or 2-butanol is between 20% (w/v) and 45% (w/v) of the total batch. Most preferably, the fraction of co-substrate in the case of isopropanol or 2-butanol is between 30% (w/v) and 40% (w/v) of the total batch.

If glucose is used as co-substrate, the composition preferably contains glucose in a concentration of 20% (w/v) to 65% (w/v) based on the total batch.

A biotransformation composition of the invention preferably comprises the redox cofactor in a concentration between 10 μM and 200 μM. More preferably, a biotransformation composition of the invention preferably comprises the redox cofactor in a concentration between 20 μM and 150 μM. Most preferably, a biotransformation composition of the invention preferably comprises the redox cofactor in a concentration between 40 μM and 100 μM.

The method according to the invention is preferably carried out at a temperature of 3° C. to 70° C. More preferably, the method of the invention is carried out at a temperature from 5° C. to 50° C. Most preferably, the method of the invention is carried out at a temperature from 15° C. to 40° C.

Preferably, the method of the invention is carried out in a pH range from 5 to 9. More preferably, the method of the invention is preferably carried out in a pH range from 6 to 7.5. Preferably, the batch is buffered to keep the pH constant. Preferably, pH is controlled via a titration device coupled to a pH meter (termed the pH-stat method).

The method according to the invention is preferably carried out at a pressure of 1 mbar to 2 bar. More preferably, the method of the invention is carried out at atmospheric pressure.

The method according to the invention can in addition be carried out in the presence of an additional organic phase.

The reaction period of the method according to the invention is preferably 5 hours to 100 hours. More preferably, the reaction period is from 10 hours to 60 hour. Most preferably, the reaction period is from 15 hours to 40 hours.

Under said conditions, starting materials of the general formula (I) are >80% reacted to a chiral secondary alcohol. More preferably, starting materials of the general formula (I) are >90% reacted to a chiral secondary alcohol. Most preferably, starting materials of the general formula (I) are >93% reacted to a chiral secondary alcohol.

The method of the invention makes it possible for the first time to produce, from starting materials of the general formula (I), a chiral secondary alcohol at yields >90% and an ee >99% at high space-time yield (starting material metered >20% w/v, 24 h reaction period) with recovery of the enzyme (recycling at least four times), with simultaneously the metered addition of CR-containing cells, expressed in dry biomass of fermenter cells, being no more than 1% (w/v) of the batch volume. The observed efficiency with simultaneous possibility of enzyme recovery and the space-time yields of the biotransformation method according to the invention which are unexpectedly high for a biotransformation are not to be expected from the prior art. In particular, it is surprising that even in an aqueous-alcoholic solution the majority of the alcohol dehydrogenase used is successfully separated off from the reaction solution by adsorption and with unrestricted activity. It is also surprising that just the presence of the adsorbent is sufficient to stabilize the enzyme activity during the reaction.

The product is isolated by known methods. Preferably, the enzyme associated with the adsorbent is first separated off from the reaction mixture for reuse, preferably by filtration, sedimentation or centrifugation. From the remaining reaction batch, the product can then be isolated directly, preferably by distillation or by extraction with a water-immiscible organic solvent. In the direct distillation, the desired end product is obtained. The end product is typically obtained in a yield >70%. More preferably, the yield is >80%. Most preferably, the yield is >90%. In each case, the yield is based on the amount of starting material (I) used. The end product has an enantiomeric excess of preferably ee >90%. More preferably, the end product has an enantiomeric excess ee >97%. Most preferably, the end product has an enantiomeric excess ee=100%.

The alternative isolation of the product from the reaction mixture by extraction can proceed discontinuously (batchwise) or continuously. As is known to those skilled in the art, a suitable temperature is set in this case in such a manner that an optimum extraction of the product from the aqueous phase is ensured. Preferably, the extraction proceeds at a temperature of 10 to 70° C.

Suitable organic solvents are all water-immiscible solvents which can extract a compound of the formula (II) or (III) from an aqueous phase. Preferably, organic solvents selected from the group of esters, ethers, alkanes, aromatics and chlorinated hydrocarbons are used. More preferably, solvents such as ethyl acetate, methyl acetate, propyl acetate, isopropyl acetate, butyl acetate, tert-butyl acetate, diethyl ether, diisopropyl ether, dibutyl ether and methyl tert-butyl ether (MTBE), pentane, hexane, heptane, toluene, methylene chloride, chloroform or mixtures thereof are used. Most preferably, the solvents are selected from MTBE, ethyl acetate, butyl acetate and methylene chloride.

After removal of the organic extraction phase, it is preferably worked up by distillation, enrichment of the reaction product being achieved and partial to complete removal of byproducts from the extraction solvent being effected and the extraction solvent being able to be used again for the extraction. By purifying the organic extraction solution containing the crude product, for example by means of fine distillation, the desired end product is obtained. The end product is typically obtained in a yield >70%. More preferably, the yield is >80%. Most preferably, the yield is >90%. In each case, the yield is based on the amount of starting material (I) used. Preferably, the product has an enantiomeric excess of ee >90%. Most preferably, the product has an enantiomeric excess of ee >97%. Most preferably, the product has an enantiomeric excess of ee=100%.

The examples hereinafter serve to describe the invention:

### 1ST EXAMPLE

### Production of Carbonyl Reductases by Fermentation

The enzyme LB-ADH, its gene and the recombinant production of LB-ADH in E. coli are disclosed in EP796914. The plasmid pADH-1 transformed into E. coli is used and disclosed in EP796914. Alternatively, the enzyme can be obtained commercially from Jülich Chiral Solutions GmbH as crude extract produced from recombinant E. coli.

The enzyme T-ADH, its gene and the recombinant production of T-ADH in E. coli are disclosed in DE 102004029112 A1. The plasmid pET24a [ADH-TS] transformed into E. coli is used and disclosed in DE 102004029112 A1. Alternatively, the enzyme can be obtained commercially from Jülich Chiral Solutions GmbH as a crude extract produced from recombinant E. coli.

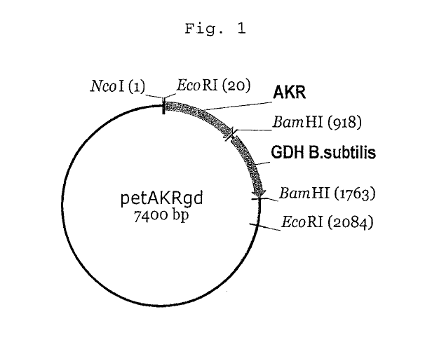

The enzyme AKR from baker's yeast is disclosed in the publicly accessible GenBank gene data bank under access number X80642 (gene name YPR1) and can be isolated according to the prior art from genomic DNA of baker's yeast. The GDH mutant GDBS-E96A is disclosed in DE 102004059376. The tandem construct pAKRgd cloned into the expression plasmid pET16B (Novagen), consisting of the AKR gene and followed by a ribosomal binding site and subsequently thereto the GDH mutant GDBS-E96A, is used (see FIG. 1 for vector map). In the cloning, the recommendations of the manufacturer of the expression vector pET16B are followed.

### Fermentation of E. coli Producing LB-ADH, T-ADH and AKR/GDH:

### Production of an Inoculum for the Fermentation:

1. Preculture of E. coli transformed by the plasmid pADH-1 (enzyme LB-ADH), or pET24a[ADH-TS] (enzyme T-ADH) or pAKRgd in LBamp medium. Culture proceeds for 7 to 8 hours on an orbital shaker (Infors) at 120 rpm and 30° C.

LBamp medium contained peptone vegetable (Oxoid) 10 g/l; yeast extract (Oxoid) 5 g/l; NaCl 5 g/l and ampicillin 0.1 g/l.

2. Preculture: 100 ml of SM3 amp medium are inoculated with 1.3 ml of shake culture in a 1 l Ehrlenmeyer flask. Culture proceeds for 16-18 hours at 30° C. and 120 rpm on an orbital shaker to a cell density OD₆₀₀/ml of 7-10. 100 ml of the preculture are used to inoculate 1 l of fermenter medium.

SM3 amp medium contains peptone vegetable (Oxoid) 5 g/l; yeast extract (Oxoid) 2.5 g/l; NaCl 0.1 g/l; ammonium sulfate 5 g/l; KH₂PO₄ 3 g/l; K₂HPO₄ 12 g/l; glucose 5 g/l; MgSO₄×7H₂O 0.3 g/l; CaCl₂×2H₂O 14.7 mg/l; FeSO₄×7H₂O 2 mg/l; sodium citrate×2H₂O 1 g/l; vitamin B1 5 mg/l; trace element mix 1 ml/l and ampicillin 0.1 g/l.

The trace element mix have the composition H₃BO₃ 2.5 g/l; CoCl₂.6H₂O 0.7 g/l; CuSO₄.5H₂O 0.25 g/l; MnCl₂.4H₂O 1.6 g/l; ZnSO₄.7H₂O 0.3 g/l and Na₂MoO₄.2H₂O 0.15 g/l.

The fermentations are carried out in Biostat CT fermenters from Sartorius BBI Systems GmbH. Fermentation medium is FM2 amp. The fermentation proceeds in fed-batch mode.

FM2 amp medium contains glucose 20 g/l; peptone vegetable (Oxoid) 5 g/l; yeast extract (Oxoid) 2.5 g/l; ammonium sulfate 5 g/l; NaCl 0.5 g/l; FeSO₄.7H₂O 75 mg/l; Na₃ citrate.2H₂O 1 g/l; CaCl₂.2H₂O 14.7 mg/l; MgSO₄.7H₂O 0.3 g/l; KH₂PO₄ 1.5 g/l; trace element mix 10 ml/l; vitamin B1 5 mg/l and ampicillin 0.1 g/l. The pH of the FM2 amp medium is set to 7.0 before the start of fermentation. In the fermentation of the T-ADH strain, the FM2 amp medium in addition contains 2 mM ZnSO₄.7H₂O.

1 l of FM2 amp is inoculated with 100 ml of inoculum. Fermentation temperature is 30° C. pH of the fermentation is 7.0 and is kept constant using the correction media 25% NH₄OH, or 6 N H₃PO₄. Ventilation proceeds using compressed air at a constant flow rate of 5 slpm (standard liters per minute). The oxygen partial pressure pO₂ is set to 50% saturation. The oxygen partial pressure is controlled via the stirrer speed (stirrer speed 450-1300 rpm). To control foam formation, Struktol J673 (20-25% v/v in water) is used.

In the course of the fermentation, the glucose consumption is determined by off-line glucose measurement using a glucose analyzer from YSI. As soon as the glucose concentration of the fermentation batches is approximately 5 g/l (5-6 h after inoculation), the addition of a 60% w/w glucose feed solution is started. The flow rate of the feed is selected in such a manner that during the production phase a glucose concentration of 1-5 g/l could be maintained.

The LB-ADH or T-ADH enzyme or the AKR/GDH enzymes are induced by adding IPTG (stock solution 100 mM) at a concentration of 0.4-0.8 mM as soon as the cell growth in the fermenter reaches an OD₆₀₀/ml of 50-60. The entire fermentation period is 32 hours. After termination of fermentation, the fermenter broth (dry biomass 50 g/l) is frozen in aliquots each of 100 ml.

Production of crude enzyme extracts from fermenter cells: 2 ml of cell suspension from the fermentation are centrifuged (10 min 3000 rpm at 4° C., Heraeus Fresco centrifuge). The sediment is resuspended in 1 ml of 50 mM potassium phosphate, pH 7.0, 1 mM MgCl₂ and disintegrated by two passages through a “French Press” homogenizer at 800 bar pressure.

The homogenate is centrifuged (10 min 3000 rpm at 4° C., Heraeus Fresco centrifuge). The supernatant gives a crude enzyme extract of 1 ml in volume. Determination of LB-ADH activity gives a volumetric activity of 1300 U/ml, or a specific activity of 108 U/mg of protein in the crude extract. Determination of the T-ADH activity gives a volumetric activity of 36 U/ml, or a specific activity of 3 U/mg of protein in the crude extract. Determination of AKR activity gives a volumetric activity of 22 U/ml, or a specific activity of 1.8 U/mg of protein in the crude extract. Determination of GDH activity gives a volumetric activity of 71 U/ml, or a specific activity of 5.9 U/mg of protein in the crude extract.

### Spectrophotometric Determination of LB-ADH and AKR Activity:

The measurement batch of 1 ml volume for the photometric determination of LB-ADH activity is composed of measurement buffer (0.1 M potassium phosphate, pH 7.0, 0.1 M NaCl, 1 mM MgCl₂), 3 μl of substrate ethyl 4-Cl-acetoacetate, 0.2 mM NADPH and LB-ADH-containing cell extract. The measurement temperature is 25° C. The reaction is started by adding the LB-ADH cell extract and the decrease in extinction owing to consumption of NADPH is measured at a wavelength of 340 nm (extinction coefficient of NADPH: ε=0.63×10⁴ l×Mol⁻¹×cm⁻¹). One unit of LB-ADH or AKR activity is defined as the consumption of 1 μmol of NADPH/min. under test conditions.

### Spectrophotometric Determination of T-ADH Activity:

The measurement batch of 1 ml volume for the photometric determination of T-ADH activity is composed of measurement buffer (0.1 M potassium phosphate, pH 7.0, 0.1 M NaCl, 1 mM MgCl₂), 3 μl of substrate acetone, 0.2 mM NADPH and T-ADH-containing cell extract. The measurement temperature is 25° C. The reaction is started by adding the T-ADH cell extract and the decrease in extinction owing to the consumption of NADPH is measured at a wavelength of 340 nm (extinction coefficient of NADPH: ε=0.63×10⁴ l×Mol⁻¹×cm^{31 1}). One unit of T-ADH activity is defined as the consumption of 1 μmol of NADPH/min. under test conditions. GDH activity is determined as described in DE102004059376 (example 3).

To determine the specific activity, the protein concentration of the cell extracts is determined in a known manner using the “BioRad protein assay” from BioRad.

### 2ND EXAMPLE

### Production of methyl (R)-3-hydroxybutyrate from methyl acetoacetate by Biotransformation Using LB-ADH Cells

1st batch: The reaction batch is composed of 20 L (20.4 kg) of methyl acetoacetate (AcMe), 20 L (15.7 kg) of isopropanol, 1 kg of Celite®, 3 L of LB-ADH cells (fermenter broth as described in the 1st example), 50 μM NADP and 7 L of KPi buffer. The composition of KPi buffer is 0.1 M potassium phosphate, pH 7.0, 0.1 M NaCl, 1 mM MgCl₂. The reaction batch is stirred at 30° C. in a 100 L reaction vessel. At various time points, 0.1 ml samples of the reaction batch are taken, extracted with 1 ml of MTBE and analyzed by chiral GC. After 24 hours, the batch is filtered by pressure filtration through a steel vacuum filter from Seitz. In the filtrate the reaction conversion rate of the AcMe used is 94.4%. The enantiomeric excess ee of the product methyl (R)-3-hydroxybutyrate is 100%. The Celite®-enzyme filter cake is returned to the reaction vessel for the 2nd batch.

The reaction is analyzed by chiral GC, with use being made of a gas chromatograph 6890N from Agilent, equipped with a Chiraldex™ G-TA from Astec (20 m×0.32 mm) for the chiral separation.

For the gas-chromatographic separation, a temperature gradient of 65° C.-170° C. having a gradient slope of 10° C./min is set. Retention times under these conditions are:
Methyl (S)-3-hydroxybutyrate: 5.9 min.
Methyl (R)-3-hydroxybutyrate: 6.5 min.
Methyl acetoacetate: 11.6 min.

2nd batch: The reaction batch is composed of 20 L (20.4 kg) of methyl acetoacetate (AcMe), 20 L (15.7 kg) of isopropanol, the Celite®-enzyme filter cake recovered from the 1st batch, 450 ml of LB-ADH cells (fermenter broth as described in the 1st example), 50 μM NADP and 9.8 L of KPi buffer. The reaction batch is stirred at 30° C. At various time points, 0.1 ml samples of the reaction batch are taken, extracted with 1 ml of MTBE and analyzed by chiral GC. After 24 h the batch is filtered (see 1st batch). In the filtrate, the reaction conversion rate of the AcMe used is 94.3%. The enantiomeric excess ee of the product methyl (R)-3-hydroxybutyrate is 100%. The Celite®-enzyme filter cake is returned to the reaction vessel for the 3rd batch.

3rd batch: The reaction batch is composed of 20 L (20.4 kg) of methyl acetoacetate (AcMe), 20 L (15.7 kg) of isopropanol, the Celite®-enzyme filter cake recovered from the 2nd batch, 450 ml of LB-ADH cells (fermenter broth as described in the 1st example), 50 μM NADP and 9.8 L of KPi buffer. The reaction batch is stirred at 30° C. At various time points, 0.1 ml samples of the reaction batch are taken, extracted with 1 ml of MTBE and analyzed by chiral GC. After 24 h the batch is filtered (see 1st batch). In the filtrate, the reaction conversion rate of the AcMe used is 94.6%. The enantiomeric excess ee of the product methyl (R)-3-hydroxybutyrate is 100%. The Celite®-enzyme filter cake is returned to the reaction vessel for the 4th batch.

4th batch: The reaction batch is composed of 20 L (20.4 kg) of methyl acetoacetate (AcMe), 20 L (15.7 kg) of isopropanol, the Celite®-enzyme filter cake recovered from the 3rd batch, 450 ml of LB-ADH cells (fermenter broth as described in the 1st example), 50 μM NADP and 9.8 L of KPi buffer. The reaction batch is stirred at 30° C. At various time points, 0.1 ml samples of the reaction batch are taken, extracted with 1 ml of MTBE and analyzed by chiral GC. After 24 h the batch is filtered (see 1st batch). In the filtrate, the reaction conversion rate of the AcMe used is 94.5%. The enantiomeric excess ee of the product methyl (R)-3-hydroxybutyrate is 100%.

The filtrates obtained from the four batches are combined (179.7 kg) and the product methyl (R)-3-hydroxybutyrate is obtained by distillation. In this process first volatile components are distilled off in a sieve-tray distillation apparatus (temperature up to 100° C., vacuum up to 150 mbar), followed by a fine distillation (temperature up to 120° C., vacuum up to 120 mbar). The yield of methyl (R)-3-hydroxybutyrate is 63 kg (77.2% yield based on a total of 81.6 kg of AcMe used).

### 3RD EXAMPLE

### Production of (R)-1-acetoxy-2-propanol from acetoxy-acetone by Biotransformation with LB-ADH Cells

1st batch: The reaction batch is composed of 20 L (41.5 kg) of acetoxyacetone, 20 L (15.7 kg) of isopropanol, 1 kg of Celite®, 10 L of LB-ADH cells, 50 μM NADP and 1 L of KPi buffer. The composition of KPi buffer is 0.1 M potassium phosphate, pH 7.0, 0.1 M NaCl, 1 mM MgCl₂. The reaction batch is stirred at 30° C. At various time points, 0.1 ml samples of the reaction batch are taken, extracted with 1 ml of MTBE and analyzed by chiral GC. After 24 hours, the batch is filtered (see 2nd example). In the filtrate the reaction conversion rate of the acetoxyacetone used is 93.9%. The enantiomeric excess ee of the product (R)-1-acetoxy-2-propanol is 100%.

The reaction is analyzed by chiral GC, as disclosed in Co10503, with use being made of a gas chromatograph 6890N from Agilent, equipped with a CP-Chirasil-Dex-CB column from Varian (25 m×0.25 mm) for the chiral separation.

For the gas-chromatographic separation, a temperature gradient of 100° C.-140° C. having a gradient slope of 2° C./min is set, followed by a temperature gradient of 140° C.-170° C. having a gradient slope of 10° C./min. Retention times under these conditions are: Acetoxyacetone: 4.4 min.
(R)-1-Acetoxy-2-propanol: 6.2 min.
(S)-1-Acetoxy-2-propanol: 6.4 min.
(R)-2-Acetoxy-1-propanol: 7.8 min.
(S)-2-Acetoxy-1-propanol: 8.7 min.

2nd batch: The reaction batch is composed of 20 L (41.5 kg) of acetoxyacetone, 20 L (15.7 kg) of isopropanol, the Celite®-enzyme filter cake recovered from the 1st batch, 1.5 L of LB-ADH cells (fermenter broth as described in the 1st example), 50 μM NADP and 8 L of KPi buffer. The reaction batch is stirred at 30° C. At various time points, 0.1 ml samples of the reaction batch are taken, extracted with 1 ml of MTBE and analyzed by chiral GC. After 24 hours, the batch is filtered (see 2nd example). In the filtrate the reaction conversion rate of the acetoxyacetone used is 95.0%. The enantiomeric excess ee of the product (R)-1-acetoxy-2-propanol is 100%. The Celite®-enzyme filter cake is returned to the reaction vessel for the 3rd batch.

3rd batch: The reaction batch is composed of 20 L (41.5 kg) of acetoxyacetone, 20 L (15.7 kg) of isopropanol, the Celite®-enzyme filter cake recovered from the 2nd batch, 1.5 L of LB-ADH cells (fermenter broth as described in the 1st example), 50 μM NADP and 8 L of KPi buffer. The reaction batch is stirred at 30° C. At various time points, 0.1 ml samples of the reaction batch are taken, extracted with 1 ml of MTBE and analyzed by chiral GC. After 24 h, the batch is filtered (see 2nd example). In the filtrate the reaction conversion rate of the acetoxyacetone used is 97.1%. The enantiomeric excess ee of the product (R)-1-acetoxy-2-propanol is 100%. The Celite®-enzyme filter cake is returned to the reaction vessel for the 4th batch.

4th batch: The reaction batch is composed of 20 L (41.5 kg) of acetoxyacetone, 20 L (15.7 kg) of isopropanol, the Celite®-enzyme filter cake recovered from the 3rd batch, 1.5 L of LB-ADH cells (fermenter broth as described in the 1st example), 50 μM NADP and 8 L of KPi buffer. The reaction batch is stirred at 30° C. At various time points, 0.1 ml samples of the reaction batch are taken, extracted with 1 ml of MTBE and analyzed by chiral GC. After 24 hours, the batch is filtered (see 2nd example). In the filtrate the reaction conversion rate of the acetoxyacetone used is 97.5%. The enantiomeric excess ee of the product (R)-1-acetoxy-2-propanol is 100%.

The filtrates obtained from the four batches are combined (183.8 kg) and distilled in vacuum to remove volatile reaction products (isopropanol, acetone) and remaining water from the crude product.

The yield of (R)-1-acetoxypropanol is 74.4 kg (86.5% yield based on in total 86 kg of acetoxyacetone used).

### 4TH EXAMPLE

### Production of methyl (S)-3-hydroxybutyrate from methyl acetoacetate by Biotransformation with T-ADH Cells

1st batch: The reaction batch is composed of 40 ml (40.8 g) of methyl acetoacetate (AcMe), 40 ml (31.4 g) of isopropanol, 2 g of Celite®, 6 ml of T-ADH cells (fermenter broth as described in the 1st example), 50 μM NADP, 2 ml of glycerol and 12 ml of KPi buffer. The composition of KPi buffer is 0.1 M potassium phosphate, pH 7.0, 0.1 M NaCl, 1 mM MgCl₂. The reaction batch is stirred at 30° C. in a 100 ml reaction vessel. At various time points, 0.1 ml samples of the reaction batch are taken, extracted with 1 ml of MTBE and analyzed by chiral GC (see 2nd example). After 24 hours, the batch is filtered (vacuum filtration through a porcelain frit). In the filtrate the reaction conversion rate of the AcMe used is 92.1%. The enantiomeric excess ee of the product methyl (S)-3-hydroxybutyrate is 100%. The Celite®-enzyme filter cake is returned to the reaction vessel for the 2nd batch.

2nd batch: The reaction batch is composed of 40 ml (40.8 g) of methyl acetoacetate (AcMe), 40 ml (31.4 g) of isopropanol, the Celite®-enzyme filter cake recovered from the 1st batch, 1.5 ml of T-ADH cells (fermenter broth as described in the 1st example), 50 μM NADP, 2 ml of glycerol and 16 ml of KPi buffer. The reaction batch is stirred at 30° C. At various time points, 0.1 ml samples of the reaction batch are taken, extracted with 1 ml of MTBE and analyzed by chiral GC. After 24 h, the batch is filtered (see 1st batch). In the filtrate the reaction conversion rate of the AcMe used is 92.2%. The enantiomeric excess ee of the product methyl (S)-3-hydroxybutyrate is 100%. The Celite®-enzyme filter cake is returned to the reaction vessel for the 3rd batch.

3rd batch: The reaction batch is composed of 40 ml (40.8 g) of methyl acetoacetate (AcMe), 40 ml (31.4 g) of isopropanol, the Celite®-enzyme filter cake recovered from the 2nd batch, 1.5 ml of T-ADH cells (fermenter broth as described in the 1st example), 50 μM NADP, 2 ml of glycerol and 16 ml of KPi buffer. The reaction batch is stirred at 30° C. At various time points, 0.1 ml samples of the reaction batch are taken, extracted with 1 ml of MTBE and analyzed by chiral GC. After 24 hours, the batch is filtered (see 1st batch). In the filtrate the reaction conversion rate of the AcMe used is 92.6%. The enantiomeric excess ee of the product methyl (S)-3-hydroxybutyrate is 100%.

4th batch: The reaction batch is composed of 40 ml (40.8 g) of methyl acetoacetate (AcMe), 40 ml (31.4 g) of isopropanol, the Celite®-enzyme filter cake recovered from the 3rd batch, 1.5 ml of T-ADH cells (fermenter broth as described in the 1st example), 50 μM NADP, 2 ml of glycerol and 16 ml of KPi buffer. The reaction batch is stirred at 30° C. At various time points, 0.1 ml samples of the reaction batch are taken, extracted with 1 ml of MTBE and analyzed by chiral GC. After 24 h, the batch is filtered (see 1st batch). In the filtrate the reaction conversion rate of the AcMe used is 92.8%. The enantiomeric excess ee of the product methyl (S)-3-hydroxybutyrate is 100%.

### 5TH EXAMPLE

### Production of ethyl (S)-4-chloro-3-hydroxybutyrate from ethyl 4-Cl-acetoacetate by Biotransformation with LB-ADH Cells

Batch without Celite®: The reaction batch is composed of 20 ml (24.3 g) of ethyl 4-Cl-acetoacetate (4Cl-ACE), 20 ml of n-butyl acetate, 40 ml (31.4 g) of isopropanol, 4 ml of LB-ADH cells (fermenter broth as described in the 1st example), 50 μM NADP, and 16 ml of KPiC buffer. The composition of KPiC buffer is 0.1 M potassium phosphate, pH 7.0, 0.1 M NaHCO₃, 1 mM MgCl₂. The reaction batch is stirred at room temperature in a 100 ml reaction vessel. At various time points, 0.1 ml samples of the reaction batch are taken, extracted with 1 ml of MTBE and analyzed by chiral GC. After 24 hours, the batch is filtered (vacuum filtration via a frit). In the filtrate, the reaction conversion rate of the 4Cl-ACE used is 57.3%. The enantiomeric excess ee of the product ethyl (S)-4-chloro-3-hydroxybutyrate (S-CHBE) is 100%. The time course of the reaction is shown in table 1.

For analysis of the reaction by chiral GC, a gas chromatograph 6890N from Agilent is used, equipped with a Chiraldex™ G-TA column from Astec (20 m×0.32 mm) for chiral separation.

For the gas-chromatographic separation, a temperature gradient of 105° C.-150° C. is established having a gradient slope of 10° C./min. Retention times under these conditions are:
Ethyl 4-Cl-acetoacetate: 4.5 min.
Ethyl (R)-4-Cl-3-hydroxybutyrate: 5.2 min.
Ethyl (S)-4-Cl-3-hydroxybutyrate: 5.7 min.

Batch A with Celite®: The reaction batch is composed of 20 ml (24.3 g) of 4Cl-ACE, 20 ml of n-butyl acetate, 40 ml (31.4 g) of isopropanol, 2 g of Celite®, 4 ml of LB-ADH cells (fermenter broth as described in the 1st example), 50 μM NADP and 16 ml of KPiC buffer. The reaction batch is stirred at room temperature in a 100 ml reaction vessel. At various time points, 0.1 ml samples of the reaction batch are taken, extracted with 1 ml of MTBE and analyzed by chiral GC. After 24 h, the batch is filtered (vacuum filtration via a frit). In the filtrate, the reaction conversion rate of the 4Cl-ACE used is 100%. The enantiomeric excess ee of the product S-CHBE is 100%. The time course of the reaction is shown in table 1.

A comparison of the reaction courses illustrated in tab. 1 shows that the LB-ADH enzyme is stabilized by the Celite® addition and a biotransformation having a significantly higher space-time yield is thereby made possible.

TABLE 1
Reaction course of S-CHBE synthesis with LB-
ADH with and without addition of Celite ®

| Batch without Celite ® | | Batch with Celite ® | |
|---|---|---|---|
| Time | Conversion rate of | Time | Conversion rate of |
| (h) | 4Cl-ACE to S-CHBE | (hours) | 4Cl-ACE to S-CHBE |
| 0 | 0.4 | 0 | 0.9 |
| 2 | 10.8 | 2 | 40 |
| 5 | 31.6 | 4 | 69.8 |
| 7 | 42.2 | 21 | 99.9 |
| 24 | 57.3 | 23 | 99.9 |

Reuse of the Celite®-LB-ADH filter cake: The batch with Celite® is repeated three times, with in each case the Celite®-LB-ADH filter cake from the preceding batch being reused and in addition in each case 2 ml of fresh fermenter cells being added. In the filtrate the reaction conversion rate of the 4Cl-ACE used is in each case 100%. The enantiomeric excess ee of the product S-CHBE is 100%.

Batch B with Celite®: The reaction batch is composed of 40 ml (48.6 g) of 4Cl-ACE, 15 ml of n-butyl acetate, 25 ml (19.6 g) of isopropanol, 2 g of Celite®, 10 ml of LB-ADH cells (fermenter broth as described in the 1st example), 50 μM NADP, and 10 ml of KPiC buffer. The reaction batch is stirred at room temperature in a 100 ml reaction vessel. At various time points, 0.1 ml samples of the reaction batch are taken, extracted with 1 ml of MTBE and analyzed by chiral GC. After 24 hours, the batch is filtered (vacuum filtration via a frit). In the filtrate, the reaction conversion rate of the 4Cl-ACE used is 100%. The enantiomeric excess ee of the product S-CHBE is 100%.

### 6TH EXAMPLE

### Production of (S)-2-hexanol from 2-hexanone by Biotransformation with T-ADH Cells

1st batch: The reaction batch is composed of 15 ml (12.2 g) of 2-hexanone, 50 ml (39.3 g) of isopropanol, 2 ml of T-ADH cells (fermenter broth as described in the 1st example), 50 μM NADP, 2 g of Celite®, and 33 ml of KPi buffer. The composition of KPi buffer is 0.1 M potassium phosphate, pH 7.0, 0.1 M NaCl, 1 mM MgCl₂. The reaction batch is stirred at 30° C. in a 100 ml reaction vessel.

At various time points, 0.1 ml samples of the reaction batch are taken, extracted with 1 ml of ethyl acetate, and analyzed by chiral GC. After 24 hours, the reaction conversion rate of the 2-hexanone used is 80%. The enantiomeric excess ee of the product (S)-2-hexanol is 100%.

For analysis of the reaction by chiral GC, use is made of a gas chromatograph 6890N from Agilent, equipped with a Chiraldex™ G-TA column from Astec (10 m×0.32 mm) for the chiral separation.

For the gas-chromatographic separation, a temperature gradient of 50° C.-70° C. having a gradient slope of 5° C./min is set, followed by 70° C.-80° C. having a gradient slope of 20° C./min. Retention times under these conditions are:
2-Hexanone: 4.30 min.
(R)-2-hexanol: 1.88 min.
(S)-2-hexanol: 2.22 min.

Reuse of the Celite®-T-ADH filter cake: The 1st batch is repeated three times, in each case the Celite®-T-ADH filter cake from the preceding batch being reused and in addition in each case 0.3 ml of fresh fermenter cells being added. In the filtrate the reaction conversion rate of the 2-hexanone used is between 79 and 83%. The enantiomeric excess ee of the product (S)-2-hexanol is in each case 100%.

### 7TH EXAMPLE

### Production of (S)-3-hydroxybutyraldehyde dimethyl acetal (S-HBDMA) from acetylacetaldehyde dimethyl acetal (AADMA) by Biotransformation with AKRgd Cells

1st batch: The reaction batch is composed of 5 ml (5 g) of acetylacetaldehyde dimethyl acetal (AADMA), 8 ml of AKRgd cells (fermenter broth as described in the 1st example), 50 μM NADP, 2 g of Celite® and 85 ml of KpiG buffer. The composition of KpiG buffer is 0.1 M potassium phosphate, pH 7.0, 0.1 M NaCl, 1 M glucose. The reaction batch is stirred at 30° C. in a 100 ml reaction vessel. The pH of 7.0 is kept constant by a titrator (“TitroLine alpha” from Schott), via which 10 M KOH is added.

At various time points, 0.1 ml samples of the reaction batch are taken, extracted with 1 ml of MTBE and analyzed by chiral GC. After 24 hours, the reaction conversion rate of the AADMA used is 93.4%. The enantiomeric excess ee of the product S-HBDMA is 100%.

For analysis of the reaction by chiral GC, use is made of a gas chromatograph 6890N from Agilent, equipped with a CP-Chirasil-Dex-CB column from Varian (25 m×0.25 mm) for the chiral separation.

For the gas-chromatographic separation, a temperature gradient of 100° C.-140° C. having a gradient slope of 2° C./min followed by 140° C.-170° C. having a gradient slope of 10° C./min is established. Retention times under these conditions are:
Acetylacetaldehyde dimethyl acetal: 5.18 min.
(S)-3-hydroxybutyraldehyde dimethyl acetal: 7.42 min.
(R)-3-hydroxybutyraldehyde dimethyl acetal: 7.66 min.

Reuse of the Celite®-AKRgd filter cake: The 1st batch is repeated three times, with in each case the Celite®-AKRgd filter cake from the preceding batch being reused and in addition in each case 2 ml of fresh fermenter cells being added. In the filtrate the reaction conversion rate of the AADMA used is between 93 and 95%. The enantiomeric excess ee of the product (S)-HBDMA is in each case 100%.

### 8TH EXAMPLE (COMPARATIVE EXAMPLE)

### Reuse of LB-ADH After Extraction of the Reaction Batch in the Production of methyl (R)-3-hydroxybutyrate from methyl acetoacetate

1st batch: The reaction batch is composed of 40 ml (40.8 g) of methyl acetoacetate (AcMe), 40 ml (31.4 g) of isopropanol, 6 ml of LB-ADH cells (fermenter broth as described in the 1st example), 50 μM NADP and 14 ml of KPi buffer. The composition of KPi buffer is 0.1 M potassium phosphate, pH 7.0, 0.1 M NaCl, 1 mM MgCl₂. The composition is equivalent to the biotransformation from the 2nd example (1st batch), but without addition of Celite®. The reaction batch is stirred at 30° C. in a 100 ml reaction vessel. At various time points, 0.1 ml samples of the reaction batch are taken, extracted with 1 ml of MTBE and analyzed by chiral GC (see 2nd example). After 24 h the batch is terminated. The reaction conversion rate of the AcMe used is 96.2%. The enantiomeric excess ee of the product methyl (R)-3-hydroxybutyrate is 100%.

The reaction batch is extracted three times each time with 100 ml of MTBE and the enzyme-containing aqueous phase is recovered. The volume of the aqueous phase is 14 ml.

2nd batch: The reaction batch is composed of 40 ml (40.8 g) of methyl acetoacetate (AcMe), 40 ml (31.4 g) of isopropanol, 14 ml of LB-ADH-containing aqueous phase after the MTBE extraction of the 1st batch, 0.9 ml of LB-ADH cells (fermenter broth as described in the 1st example, is equivalent to the amount of cells with which the 2nd batch in the 2nd example is restored), 50 μM NADP and 5.1 ml of KPi buffer. The reaction batch is stirred at 30° C. in a 100 ml reaction vessel. At various time points, 0.1 ml samples of the reaction batch are taken, extracted with 1 ml of MTBE and analyzed by chiral GC (see 2nd example). After 24 h, the batch is terminated. The reaction conversion rate of the AcMe used is 50.9%. The enantiomeric excess ee of the product methyl (R)-3-hydroxybutyrate is 100%.

This comparative example shows that reuse of the enzyme after product extraction with MTBE leads to an incomplete reaction. As shown in the 2nd example, in contrast thereto, the enzyme is stabilized by adsorption to Celite® under otherwise identical conditions in such a manner that on reuse a virtually complete reaction conversion rate is achieved and the enzyme is available for further reaction cycles.

1. A method of producing a chiral secondary alcohol, the method comprising:
a) reacting a biotransformation composition comprising:
i) a ketone of the formula (I), 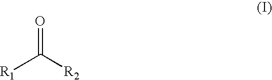
R₁ and R₂ being different and each being an organic radical;
ii) an oxidoreductase; and
iii) a co-substrate;
and an adsorbent to form a chiral secondary alcohol, the adsorbent being associated with the oxidoreductase; and
b) separating the adsorbent associated with the oxidoreductase from the biotransformation composition after completion of the reaction.

2. The method of claim 1, wherein the adsorbent comprises a solid that keeps the oxidoreductase in an active form during the biotransformation reaction and retains the oxidoreductase in a filter cake in active form after termination of the biotransformation and separation of the adsorbent from the reaction batch.

3. The method of claim 1 wherein the adsorbents are selected from the group consisting of aluminum oxide, silica gel, Mg silicate (for example Florisil®), bentonite, Celite®, XAD, Dowex®, Amberlite®, Sepharose®, Sephadex®, Superose®, and cellulose such adsorbants being suitable for hydrophobic or electrostatic interaction.

4. The method of claim 3, wherein the adsorbent is selected from the group consisting of XAD, Florisil®, silica gel, and Celite®.

5. The method of claim 4, wherein the adsorbent is Celite®.

6. The method of claims 1, wherein the oxidoreductase associated with the adsorbent is separated off by filtration, sedimentation or centrifugation for reuse of the reaction mixture.

7. The method of claims 1, wherein the chiral secondary alcohol is a compound of the formula (II) or (III) 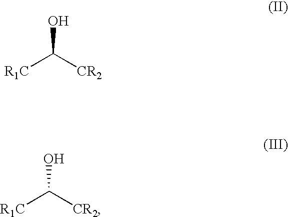
R₁ and R₂ being different from one another and being an organic radical.

8. The method of claim 7, wherein R₁ and R₂ are different organic radicals having 1-20 carbon atoms with one or more carbon atoms of the radicals R₁ or R₂ being optionally replaced by:
i) atoms selected from the group consisting of B, N, O, Si, P, S, F, Cl, Br, and I;
ii) optionally substituted C₃-C₈-cycloalkyl, C₆-C₂₀-aryl, C₅-C₂₀-heteroaryl
iii) silyl radicals; or
iv) CN, NH₂, NO or NO₂.

9. The method of claim 8, wherein R₁ and R₂ are unbranched or branched C₁-C₂₀-alkyl, C₂-C₂₀-alkenyl, C₂-C₂₀-alkynyl, C₃-C₈-cycloalkyl, C₆-C₂₀-aryl or C₅-C₂₀-heteroaryl radicals, with one or more carbon atoms of the radicals R₁ or R₂ being optionally replaced by:
i) atoms selected from the group consisting of B, N, O, Si, P, S, F, Cl, Br, and I;
ii) optionally substituted C₃-C₈-cycloalkyl, C₆-C₂₀-aryl, C₅-C₂₀-heteroaryl
iii) silyl radicals; or
iv) CN, NH₂, NO or NO₂.

10. The method of claim 1, wherein the oxidoreductase is a carbonyl reductase having S or R specificity.

11. The method of claim 10, wherein the biotransformation composition comprises a redox cofactor selected from compounds of the group consisting of NAD, NADP, NADH, NADPH and salts thereof.

12. The method of claim 11, wherein the carbonyl reductase is an alcohol dehydrogenase and the co-substrate is an alcohol.

13. The method of claim 12, wherein the alcohol is isopropanol or 2-butanol.

14. The method of claim 1, wherein starting materials of the general formula (I) are >80% reacted to a chiral secondary alcohol.

15. The method of claim 1, wherein the adsorbent which has been separated off and is associated with the oxidoreductase is used in a second reaction cycle.

16. The method of claim 15, wherein up to 20 reaction cycles are carried out using adsorbent separated off in a previous reaction cycle.

17. The method of claims 1 wherein the chiral secondary alcohol is extracted by distillation or by means of a water-immiscible organic solvent after being separated from the absorbent.

18. A biotransformation composition comprising:
fermenter cells containing a CR enzyme, an adsorbent;
a compound of the formula (I): 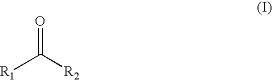
wherein R₁ and R₂ are a different organic radical;
a redox cofactor selected from the compounds NAD, NADH, NADP, NADPH and salts thereof,
a co-substrate selected from the group isopropanol, 2-butanol and glucose; and
a GDH as a cofactor-regenerating enzyme if glucose is used as the co-substrate.

19. The composition of claim 18 wherein an amount between 1% (v/v) and 40% (v/v) of a fermentation medium is present, the fermentation medium containing fermenter cells and having a biomass fraction of 0.05-2% (w/v).

20. The composition of claim 18 wherein:
the adsorbent is present in an amount of 0.1-10% (w/v) of the biotransformation composition
the compound having formula (I) is present in an amount of 5% (w/v) to 60% (w/v) of the biotransformation composition; and
the co-substrate is present in an amount between 10 μM and 200 μM.

21. The composition of claim 18, wherein the CR enzyme is an ADH.

22. The composition of claim 18, wherein the adsorbent is Celite®.
